(19) United States
(12) Patent Application Publication
(10) Pub. No.: US 2004/0166581 A1
(43) Pub. Date: Aug. 26, 2004

(54) Highly selective molecular sensor based on dual MIP/QCM elements and a method of use thereof

(76) Inventor: Jacques Penelle, Verrieres-le-Buisson (FR)

Correspondence Address: Law Office of William B. Ritchie, 43 Jackson Street, Concord, NH, 03301, US

(21) Appl. No.: 10/730,369

(22) Filed: Dec. 8, 2003

Related U.S. Application Data
(60) Provisional application No. 60/431,869, filed on Dec. 9, 2002.

(51) Int. Cl. G01N 33/00

(52) U.S. Cl. 436/2; 422/68.1

(57) ABSTRACT

A molecular sensor for detecting small concentrations of a target molecular even in the presence of other molecules having similar shape and chemistry. Two QCM sensors are provided, both of which are covered with polymeric coatings having essentially the same chemistry. One of the QCM sensors is molecularly imprinted while the other is not. The output of the two QCM signals is compared to indicate the presence of the target molecule.

This application claims benefit of filing date of U.S. Provisional Application Serial No. 60/431,869 filed on Dec. 9, 2002 pursuant to 35 USC § 119(e).

### FIELD OF THE INVENTION

This invention relates to the field of molecular sensors based on a quartz crystal microbalance (QCM) and molecular imprinting (MIP) and a method of use thereof.

### BACKGROUND OF THE INVENTION

Molecular sensors based on a quartz crystal microbalance (QCM) are sensitive and inexpensive devices that generate a frequency shift in the quartz crystal vibration when molecules interact with its surface. To achieve high sensitivity and high selectivity to a targeted substance, the QCM's active area (that in contact with the liquid) must normally be coated with a functional layer that complexes or adsorbs the substance. Sensitivity is defined as the lowest concentration of a substance that can be detected, and selectivity is defined as the ability to distinguish one substance in the presence of similar substances. When sensitive and selective to a liquid contaminant, QCM sensors are competitive with other sensor types in terms of cost, speed or response, physical size, and other measures of practical performance. In a thickness shear mode QCM device, the active area is a metal electrode. Various QCM coatings have been discussed in the literature.

Molecular recognition is a basic biological process. Biotechnology has allowed the development of recognition molecules, which are designed to recognize and interact with a specific target or ligand. The technique known as molecular imprinting permits the construction of molecules with specific recognition properties. The most interesting aspect of this construction is that the target or ligand determines the structure of the construction. Molecular imprinting creates specific binding and catalytic sites for organic molecules in cross-linked network polymers. It involves pre-organization of polymerizable monomers around an imprinting molecule. After polymerization and removal of the imprinted molecule, the solid polymer contains binding sites, which are complementary in size and shape to the template. This cavity acts as a lock for the receptor by carefully shaped pores that amount to artificial receptors. The shape and chemistry of these pores enables them to serve as specific detectors. This concept of lock and key has been the basis of this technology. MIP has a highly specific molecular memory, and is capable of binding with the selected target molecule. MIP is more stable and robust with their highly cross-linked polymeric structure than, for example, real antibodies currently used in sensor technology. MIP may be used in harsher environments, such as bodily fluids.

Designing sensors for pollutants has always been, and still is, an important technological challenge. Sensors are customarily required to possess more sensitivity and accuracy or selectivity in the recognition of the target molecule. Therefore, although there are already sensors that use QCM, the art continuously requires new technologies and methods to improve the sensitivity and selectivity of detection. QCM sensors are useful for detecting targeted chemicals or pollutants in a monitored area or solution. Though recent, the molecular imprinting technique is known in the art to detect target molecules of pollutants. This has permitted obtaining very sensitive sensors for polycholoroaromatic contaminants for detection in picomolar concentrations with a short response time. While some selectivity has been possible using prior art apparatus, this selectivity has not been sufficient to discriminate without ambiguity between molecules of similar shape and chemistry and, in particular, when high concentrations of the antagonist have been encountered.

There is not found in the prior art a sensor that uses the combination of both QCM and MIP technologies yet ensures increased senility and selectivity to discriminate without ambiguity between molecules having similar shape and chemistry, even in the presence of high concentrations of the antagonist.

### SUMMARY OF THE INVENTION

It is an aspect of the invention to combine MIP's molecular recognition technology, which allows for the selective recognition of the targeted molecules by cross-linked polymers, with QCMs, in a novel arrangement of these elements which utilizes two sensing elements, only one of which is molecularly imprinted. The invention uses two MIP/QCM sensors where the MIP coats a cross-linked templated polymeric layer of the QCM. One sensor possesses an imprinted polymer and the other a non-imprinted polymer. Each sensor is connected to a subtracting electronic device which receives the output of each sensor and compares attracted molecules by the imprinted QCM to those of the non-imprinted QCM. The invention combines two MIP/QCM in conjunction with specific novel chemistry to fabricate thin polymeric films which have been previously disclosed and described by the inventor under U.S. patent application Ser. No. 09/915,718 filed on Jul. 26, 2001, which is incorporated by reference in its entirety. This combination or arrangement of elements allows for the obtaining of a very sensitive and selective detection of contaminants.

The QCM sensing element uses a coating of a cross-linked templated polymer layer to the QCM surface, such that cavities of molecular dimensions created in the polymeric matrix maintain some molecular memory of the original template after its physical removal. The combination of elements in the invention, including the specific chemistry used in the fabrication of polymer films, cause high selectivity and sensitivity of the sensor, reaching picomolar concentrations, and a short response time.

This aspect of the invention is not meant to be exclusive and other features, aspects, and advantages of the present invention will be readily apparent to those of ordinary skill in the art when read in conjunction with the appended claims.

### BRIEF DESCRIPTION OF THE DRAWINGS

FIG. 1 is an illustration showing the sensor system in accordance with the invention.

FIG. 2 is an illustration showing a view of a molecular imprinted polymer QCM sensor interacting with the target molecules.

### DESCRIPTION OF THE PREFERRED EMBODIMENTS

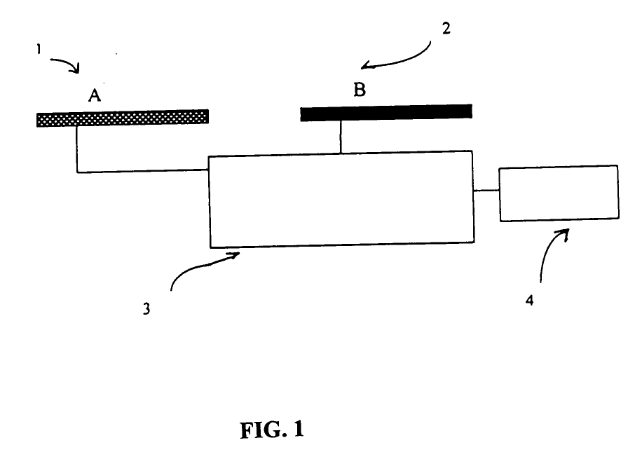

The invention is an improved molecular sensor having two sensing elements A and B as illustrated in FIG. 1. Both elements, sensor 1 and sensor 2, are a QCM covered by a polymeric coating of the same chemistry. Only sensor 1 is molecularly imprinted. A subtracting electronic apparatus 3 is connected to sensors 1, 2 and compares the output signal coming from sensor 1 and sensor 2 and measures intensity difference. An output signal is then sent from apparatus 3 to recording apparatus 4.

The invention operates as follows. The resulting signal V obtained in the presence of the target molecule is V=signal [A]−signal [B]>0, while the signal W arising in the presence of an antagonist will be W=signal [A]−signal [B]≧0. Whatever the selectivity of the system, V≧W. Recent analysis of previous experimental results suggests that in most cases, V will be much larger than W. It appears that most loss in selectivity, in previously described MIP/QCM sensors, arise from the non-selective interactions wit the polymeric matrix, and not from interactions with imprinted cavities. This has been demonstrated by analyzing the selectivity of various imprinted and non-imprinted coatings toward a range of very different molecules. A linear relationship between non-selective interactions and lipophilicity of the molecule was discovered, indicating that a significant part of the signal, even in imprinted systems, is coming from a partitioning mechanism from the aqueous solution to the lipophilic coating.

In a preferred embodiment of the invention, a microprocessor is included to process the signal sent form the MIP/QCM sensors. The microprocessor can effect the subtraction operation and record the results showing the amounts of contaminant present in the sample.

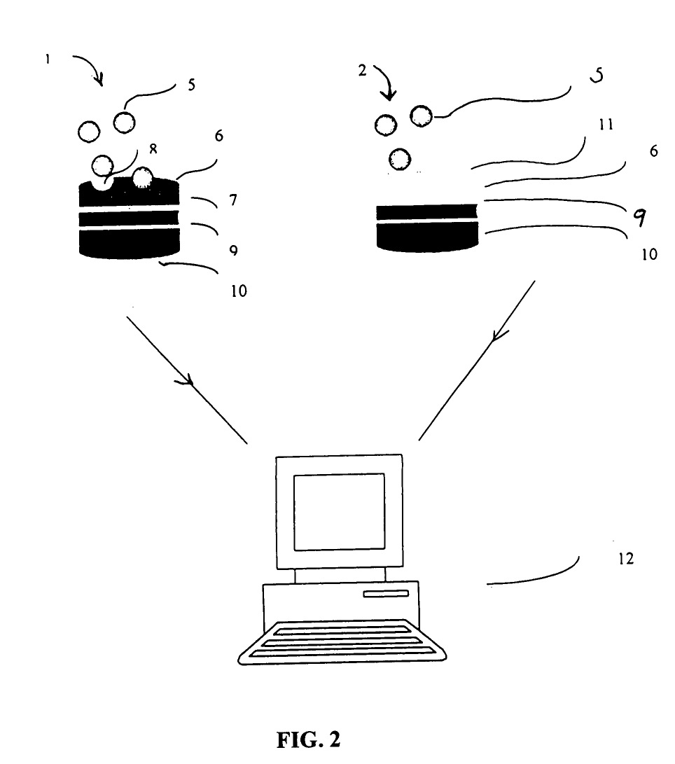

Referring next to FIG. 2, a schematic view of the QCM MIP sensors system is shown with a microprocessor 12 which serves as the subtracting apparatus 3 and recorder 4. To demonstrate the interaction in practice, the QCM sensor 1 is covered by an MIP layer 6 that has been molecularly imprinted to recognize the target molecules or analytes 5. The MIP receptor 6 consists of a layer of molecular imprinted polymer having recognition sites or cavities 8 that recognize and attracts specific target molecules 5. Another QCM sensor 2 with no molecularly imprinted recognition sites 11 is also part of the invented system. An interface 9 and transducer 10 are depicted in FIG. 2 as well. The molecules 5 that are attracted by the QCM 1 possessing the molecularly imprinted layer 8 and the molecules 5 attracted by the QCM 2 with the non-imprinted layer 11 cause a signal that is sent through the transducer 10. The signal is sent indicating the targeted molecule 5 recognized by the QCM 1 with molecular imprinted coating and the molecule attracted by the QCM 2 with no molecular imprint 11. This date is sent to a microprocessor 12 that will perform a subtracting operation and record these results.

The illustrated embodiments of the invention are intended to be illustrative only, recognizing that persons having ordinary skill in the art may construct different forms of the invention that fully fall within the scope of the subject matter disclosed herein. Other features and advantages of the invention will be apparent from the descriptions hereof and from the claims.

What is claimed is:

1. A sensor comprising:
a first quartz crystal microbalance (QCM) covered by a polymeric coating having a predetermined chemistry;
a second quartz crystal microbalance (QCM) covered by a polymeric coating consisting essentially of the same predetermined chemistry of said first QCM and wherein said second QCM is molecularly imprinted, wherein said first and second QCMs have an output corresponding to a target molecule that is to be detected;
subtracting means for comparing the output of said first QCM to the output of said second QCM to provide a signal that corresponds to the compared level of intensity between said first and second QCMs; and
recording means for recording the signal provided by said subtracting means.

2. The sensor system as claimed in claim 1 comprising a microprocessor in communication with said recording means wherein said microprocessor being programmed to process the signal stored in said recording means such that said microprocessor indicates the presence of the target molecule even when other molecules having similar shape and chemistry are also present.

3. A method for detecting at least one target molecule comprising the steps of:
providing a first and a second quartz crystal microbalance (QCM);
disposing said first and second quartz crystal microbalance (QCM) in an environment to be monitored;
coating the surface of the first QCM with a molecular imprinted polymer;
coating the surface of the second QCM with a non-imprinted polymer;
comparing the output of said QCM with the output of said QCM to provide a comparison signal;
recording the comparison signal; and
processing the comparison signal to provide an indication of the target molecule even in the presence of other molecules having similar shape and chemistry.
